(19) United States
(12) Patent Application Publication
(10) Pub. No.: US 2007/0071817 A1
(43) Pub. Date: Mar. 29, 2007

(54) Effervescent oral care compositions and method of use

(75) Inventor: Allen H. Rau, Cincinnati, OH (US)

Correspondence Address: MERCHANT & GOULD PC, P.O. BOX 2903, MINNEAPOLIS, MN, 55402-0903, US

(73) Assignee: PHYZZ, INC.

(21) Appl. No.: 11/234,312

(22) Filed: Sep. 26, 2005

(51) Int. Cl. A61K 9/46

(52) U.S. Cl. 424/466

(57) ABSTRACT

Solid effervescent compositions are described. The solid compositions include an edible acid and a carbonate salt (e.g., sodium carbonate or sodium bicarbonate). The compositions can further include one or more water soluble functional ingredients. The solid compositions can be free of insoluble particulate material so as to prevent damage to the teeth or gums. The composition can be slightly acidic to assure complete dissolution and so that its flavor does not become salty. The solid compositions can be dissolved in water to form a solution having an osmolality of 310 mOsm/kg of water or less. The resulting solution can then be contacted with the oral tissues of a user. Alternatively, the solid composition can be placed directly into the oral cavity of a user and allowed to dissolve and effervesce in the oral cavity.

### BACKGROUND

This application is related to U.S. patent application Ser. No. ______, filed ______ (Attorney Docket No. 70037.0013US01), which is incorporated by reference herein in its entirety.

### TECHNICAL FIELD

This application relates generally to oral care products and, in particular, to effervescent oral care compositions that include an effervescent acid and a carbonate salt and to the use of these compositions.

### BACKGROUND OF THE TECHNOLOGY

Various patents describe the general concept of providing a tablet or capsule that can be put in the mouth of a user for various purposes. U.S. Pat. No. 2,778,045, for example, teaches the use of a capsule that is broken by the teeth to release a dentifrice. Alternatively the dentifrice may be allowed to dissolve, followed by the use of a brush. Also suggested is the use of the capsule itself as a brush. U.S. Pat. No. 3,116,208 discloses a dental cleanser in tablet form. Calcium carbonate is mixed with sodium lauryl sulfate to bind together into a tablet that may be crushed by the teeth. The sodium lauryl sulfate is said to cause foaming upon brushing the teeth. U.S. Pat. No. 3,431,339 discloses a dental tablet for use in place of toothpaste. The tablet is said to be an intimate blend of water-soluble fluorine containing agents, polishing agents and foaming agents in a releasable matrix. Sodium lauryl sulfate is disclosed as a foaming agent.

Other patents that disclose oral care compositions are set forth below. U.S. Pat. No. 834,676 discloses formulations that may be compressed into a tablet or lozenge form. U.S. Pat. No. 975,814 discloses formulations in tablet form. U.S. Pat. No. 1,411,681 discloses a thin tablet that fits between the rows of bristles on a toothbrush. U.S. Pat. No. 1,516,398 discloses a chewing gum with a treating agent contained in a cavity in the gum. U.S. Pat. No. 3,497,590 discloses using an aliphatic aldehyde or oxyderivative thereof in a dental product such as toothpaste, tablets, chewing gum and lozenges. U.S. Pat. No. 3,518,343 discloses an effervescent tablet form cleaning the oral cavity by dissolution of the tablet in water. U.S. Pat. No. 4,308,252 discloses a tablet that can be rehydrated into a viscous paste and applied by a dental hygienist.

U.S. Pat. No. 3,629,468 to Anderson discloses a chewable mouthwash tablet. This tablet includes adipic acid and/or adipic anhydride in order to control the hygroscopicity of the product. U.S. Pat. No. 4,919,918 discloses a non-alcoholic mouthwash in the form of an effervescent tablet that includes large amounts (i.e., 38%) of spray dried flavoring agent. U.S. Pat. No. 5,464,608 discloses an effervescent composition for cleaning teeth and prophylaxis of caries. U.S. Pat. No. 6,066,335 discloses a method of making effervescent tablets in which glycerin or propylglycol is used a binder.

Four related patents disclose oral care compositions that include an insoluble silica material as an abrasive. U.S. Pat. No. 5,804,165 discloses an antiplaque oral composition using a source of carbon dioxide, silica and xylitol where the carbon dioxide comes from a bicarbonate. The effervescent tablet converts to a solid silica containing suspension in the saliva of an oral cavity. U.S. Pat. No. 5,817,294 discloses an oral dentrifice composition comprising bicarbonate and an acid in a ratio of 0.32 to 1.0 to 0.8 to 1.0. U.S. Pat. No. 5,965,110 discloses compositions comprising a carbon dioxide source and acid with silica. U.S. Pat. No. 6,086,854 discloses compositions comprising a carbon dioxide source, an acid, xylitol and precipitated amorphous silica.

U.S. Pat. No. 4,267,164 discloses an effervescent mouthwash tablet having a ratio of acid to bicarbonate of 3:2. U.S. Pat. No. 5,670,138 discloses mouth care products including effervescent tablets that contain copolymers of N-vinylpyrrolidone and acrylic acid. U.S. Pat. No. 6,254,856 discloses compositions for the removal of dental plaque that comprise various enzymes and have pH values between 6.0 and 8.0. U.S. Pat. No. 6,355,228 discloses oral care products that contain specific polypeptides. U.S. Pat. No. 6,428,770 relates to solid preparations for oral hygiene. These preparations are multilayer effervescent tablets in which one layer contains a higher level of a medicinal component. U.S. Patent Application Publication No. 2004/0126335 A1 is directed towards a method of enhancing fluoridation and mineralization of teeth by using fluoride and a phosphonate group containing polymeric mineral surface active agent. U.S. Patent Application Publication No. 2004/0223921 A1 discloses an effervescent oral care tablet including a carbon dioxide source and an acid source.

There still exists a need for solid oral care compositions which are pleasing to the user and that do not include any solid, insoluble material such as an abrasive material that can impart a gritty feeling to the composition.

### SUMMARY

According to a first embodiment, a composition is provided which comprises an effervescent acid and a carbonate salt dissolved in water, wherein the composition has an osmolality of 310 mOsm/kg of water or less.

According to a second embodiment, a method is provided which comprises: dissolving a solid composition comprising an effervescent acid and a carbonate salt in water to form an effervescent solution; and contacting the solution with oral tissue of a subject; wherein the solution has an osmolality of 310 mOsm/kg or less.

According to a third embodiment, a method is provided which comprises:

placing a solid composition comprising an effervescent acid and a carbonate salt into an oral cavity of a subject; and

allowing the composition to dissolve and effervesce in the oral cavity;

wherein the weight ratio of carbonate to acid is in the range of 1.25 to 4.5; and

wherein the composition is substantially free of insoluble particulate material.

In any of the aforementioned embodiments, exemplary effervescent acids include, but are not limited to, citric acid; fumaric acid; tartaric acid; malic acid; and adipic acid. Exemplary carbonate salts include, but are not limited to, sodium, potassium, ammonium, magnesium, and calcium organic carbonate salts. The solid composition can be in tablet form.

### DETAILED DESCRIPTION

Oral care products are currently available in many forms. Mouthwashes and fluoride rinses, for example, are typically liquids. Dentifrices are usually formulated as viscous gels, pastes or powders. Breath fresheners can be found in tablet or strip form. Whiteners are typically provided in gel or gel-on-strip forms.

Oral care compositions in tablet form provide certain advantages for delivering functional ingredients to the oral cavity, particularly to the teeth and gums. First, tablets are highly concentrated product forms and thus can carry high levels of solid ingredients. It can often be difficult to carry high levels of functional materials in liquid products as solubility limitations can cause product instability. This benefit also eliminates the need for the product to contain a high proportion of water. Thus the product can be transported very economically. Second, tablets provide an interesting sensory experience for the user. This is particularly true in the case of effervescent tablets. The gas releasing action of the effervescent products provides multisensory visual and auditory stimulation to the user.

Formulating effervescent ingestable tablets poses certain challenges. For example, the balance of acidic and carbonate components can affect the taste and the properties of the tablet. If the carbonate compounds (e.g., sodium carbonate, sodium bicarbonate, potassium bicarbonate, potassium carbonate, calcium carbonate, and/or magnesium carbonate) are present in too great an excess, the product can taste salty. Further, if these ingredients are present at too high a level the product pH will become too high for them to fully dissolve. This will cause grittiness and possibly abrasion to the teeth, gums and oral cavity surfaces. On the other hand, if the acidic materials are present in too great an excess, the product may taste too bitter. Further, a highly acidic environment can damage the teeth.

An effervescent solid composition (e.g., a tablet) that that leaves a clean feeling in the mouth and that can be used to carry a variety of functional ingredients to the oral cavity is described herein. In order to be acceptable for consumer use, this tablet will ideally dissolve completely and quickly without grittiness, have a flavor that is not too salty or acidic and that is compatible with available flavors and sweeteners, and not be abrasive to the teeth, gums or any other part of the mouth.

The solid composition comprises an effervescent acid and a carbonate salt. Exemplary effervescent acids include, but are not limited to, citric acid, fumaric acid, tartaric acid, malic acid and adipic acid. Other edible acids can also be used. Exemplary carbonate salts include, but are not limited to, sodium bicarbonate and sodium carbonate as well as potassium, ammonium, magnesium, calcium or other metal or organic carbonate salts.

As will be illustrated in following examples, the ratio of the acid component to the carbonate component can affect the performance of the product. If the ratio is too acidic, the taste will be too bitter or tart. Alternatively, if there is excess carbonate, the product will taste salty and will not fully dissolve, leaving a gritty feeling in the mouth.

In addition to the acid/carbonate salt, the composition may contain various binders, fillers and/or lubricants. These materials can be chosen from among the known materials that are used for these functions that are either water soluble or are not gritty when dispersed in water or saliva. Some non-limiting examples of these materials are polyethylene glycol, polypropylene glycol, polyvinyl alcohol, polyvinyl pyrrolidone, sodium benzoate, leucine, magnesium stearate, polyethylene glycol behenate, glyceryl behenate, sodium lauryl sulfate, sodium lauryl sulfoacetate, sorbitol, lactose, urea, sucrose stearate, starch, maltodextrin, corn syrup solids, sodium citrate, sodium sulfate, sodium chloride, sucrose, dextrates, and the like.

Excipients that modify the flavor and/or mouthfeel of the product may also be included. Non-limiting examples of these materials are sweeteners such as calcium or sodium saccharin, aspartame, acesulfame potassium, sucralose, cyclamates, sucrose, glucose, xylitol, manitol or other sugar, pectin, guar gum, gum arabic, xanthan gum, starch, maltodextrin, corn syrup solid, hydroxymethyl cellulose, hydroxypropyl cellulose, tragacinth gum, alginic acid or salts of alginic acid, and, of course, flavorants.

Flavor additives can also be used. Since the product pH will typically be somewhat acidic, flavors that are accentuated by acidity are preferred. Some examples of these types of flavors are citrus types (e.g., lemon, lime, orange grapefruit, etc.), ginger, various berries (e.g., raspberry, strawberry, blueberry, etc.) and mint types (e.g., peppermint, spearmint, wintergreen). These flavor types can be combined with other flavor additives to yield an acceptably flavored product. Exemplary combinations include, but are not limited to: spearmint/orange, cinnamon/clove/orange, and lemon/mint. A citrus flavoring can also be included to improve the overall perception of the product's taste.

Functional additives can also be incorporated into the composition. Non-limiting examples of these additives and their functions are:
Anhydrous surfactants such as sodium lauryl sulfate, sodium lauryl sulfoacetate, cocamidopropyl betaine, sodium alpha olefin sulfonate, dioctyl sodium sulfosuccinate, and sodium dodecyl benzene sulfonate. These materials can be added to the composition to generate foam. The resulting composition can then function as a dentifrice.
Anticaries ingredients such as sodium fluoride, sodium monofluorophosphate and stannous fluoride. These materials are known to help prevent tooth decay.
Bleaching agents such as carbamide peroxide (also known as urea peroxide), sodium perborate, calcium peroxide, and sodium percarbonate. These materials can whiten teeth.
Enzymes such as papain and other proteases, amylases, and lipases can be used to help remove plaque and clean the teeth.
Desensitizing agents such as strontium nitrate and potassium nitrate. These materials reduce the unpleasant stimulation caused by heat or cold felt by many people feel on their teeth.
Antimicrobial agents such as cetylpyridinium chloride and domiphen bromide. These materials reduce the bacterial population of the oral cavity.
Breath freshening ingredients such as flavoring agents (see above), chlorophyll, and the antimicrobial ingredients listed above. These materials can help reduce mouth odors by eliminating bacteria and by covering the odors with strong, typically minty, fragrances.
Other prescription medicines such as antibiotics and chlorhexidine gluconate.
Dyes used at levels that will color the foam generated by any surfactant that is incorporated in the product. This will add sensory interest to products designed for children.

Naturally, any material incorporated in this product will have to be of food or drug grade quality and must be safe for ingestion. Also, since effervescent products are chemically reactive (the acid combines with the carbonate salt to release water, carbon dioxide and the salt of the acid) by nature, it is very important that all materials used in them be essentially anhydrous. The maximum amount of moisture that, in general, can be incorporated in a well formulated effervescent product without inducing the effervescent reaction is 1%. Preferably this value is below 0.2%.

Exemplary formulations are presented below in Table 1:

| | Formulations (w/w) | | | | | | | |
|---|---|---|---|---|---|---|---|---|
| Material | A | B | C | D | E | F | G | H |
| Citric Acid | 50.0 | 37.5 | 30.0 | 30.0 | 30.0 | 20.0 | 15.0 | 15.0 |
| Sodium30.0 | 30.0 | 37.5 | 30.0 | 50.0 | 50.0 | 55.0 | 65.0 | |
| Bicarbonate | | | | | | | | |
| Mint Flavor | 5.0 | 5.0 | 5.0 | 5.0 | 5.0 | 5.0 | 5.0 | 5.0 |
| Sucralose | 0.5 | 0.5 | 0.5 | 0.5 | 0.5 | 0.5 | 0.5 | 0.5 |
| PEG-180 | 2.0 | 2.0 | 2.0 | 2.0 | 2.0 | 2.0 | 2.0 | 2.0 |
| Sodium Lauryl | 0.1 | 0.1 | 0.1 | 0.1 | 0.1 | 0.1 | 0.1 | 0.1 |
| Sulfate | | | | | | | | |
| Sorbitol | 12.4 | 24.9 | 24.9 | 32.4 | 12.4 | 22.4 | 22.4 | 12.4 |
| TOTAL | 100.0 | 100.0 | 100.0 | 100.0 | 100.0 | 100.0 | 100.0 | 100.0 |
| Carbonate/Acid | 0.6 | 0.8 | 1.25 | 1.00 | 1.67 | 2.50 | 3.67 | 4.33 |
| Ratio | | | | | | | | |
| pH (3% | 4.1 | 4.3 | 5.0 | 4.6 | 5.3 | 5.8 | 6.2 | 6.6 |
| solution) | | | | | | | | |
| Taste | Too | Slightly | OK | OK | OK | OK | Slightly | Slightly |
| | tart | Tart, OK | | | | | salty, ok | salty, ok |
| Osmolality | | 156 | | | 198 | 278 | | |
| (mOsm/kg) | | | | | | | | |

As can be seen from Table 1, compositions B, E and F had osmolality values of 156, 198 and 278 mOsm/kg, respectively. The present inventor has determined that solutions having osmolality values exceeding 310 mOsm/kg (milliosmoles per 5 kilogram) can result in damage due to cell dehydration when contacted with oral tissues. Osmolality in Table I was measured on the solutions created by dissolving 600 mg of the composition (in tablet form) in 25 ml of distilled water.

An exemplary tablet making procedure is as follows:

Add the flavor oil to the sorbitol and sodium bicarbonate;

Mix well to distribute the oil uniformly on the substrate;

Add the balance of the excipients and mix until uniform;

Press into the desired size and weight tablets (e.g., using conventional tablet making equipment).

The typical tablet size is between 0.25 grams and 1.0 gram. The tablet can typically be dissolved in 15-25 ml of water.

It should be noted that the acceptable carbonate to acid ratio can be varied somewhat depending on the flavor and possibly the sweetener combination chosen for the particular product. Also, the pH can be adjusted to optimize some flavors. For instance, citrus notes may be perceived to be more acceptable if the pH is in the lower part of the range. In addition, some mints may be better at the higher end of the pH range.

### OTHER EXAMPLES

It will be apparent to one skilled in the art that additional or alternate functional materials can be incorporated in this base product. Exemplary formulas for some of these products are shown in Table 2 below:

| | Mouthwash Type Formulations (% w/w) | | | |
|---|---|---|---|---|
| Material | Desensitizing | Fluoride | Whitening | Antimicrobial |
| Citric Acid | 30.00 | 30.00 | 30.00 | 30.00 |
| Sodium | 37.50 | 37.50 | 37.50 | 37.50 |
| Bicarbonate | | | | |
| Sodium Lauryl | 0.10 | 0.10 | 0.10 | 0.10 |
| Sulfate | | | | |
| Potassium Nitrate | 5.00 | | | |
| Sodium Fluoride | | 0.24 | | |
| Calcium Peroxide | | | 1.0 | |
| Cetylpyridinium | | | | 0.30 |
| Chloride | | | | |
| Flavor | 0.50 | 0.50 | 0.50 | 0.50 |
| Sucralose | 0.05 | 0.05 | 0.05 | 0.05 |
| PEG-180 | 2.00 | 2.00 | 2.00 | 2.00 |
| Sorbitol | 24.85 | 29.61 | 28.85 | 29.55 |
| TOTAL | 100.00 | 100.00 | 100.00 | 100.00 |

Product developers experienced in formulating tablets will recognize that excipients that will enhance the processing of this product may be added to it. In addition to the lubricants mentioned above, flow agents and adsorbents may be needed to increase the efficiency of the manufacturing operation. Materials such as fumed silica, calcium silicate, talc, and corn starch are commonly used for these purposes. Although these materials may be insoluble in water, they are of sufficiently fine particle size and softness that they will not impart grittiness or abrasivity to the product.

It should be noted that none of the examples shown above incorporate materials that could be abrasive to the teeth, settle in the mouthwash solution or have a perceptible gritty feel in the mouth. Even when insoluble materials such as calcium carbonate are used, the pH of the product can be adjusted so that the material becomes soluble. This is done without dropping the pH to a level that could be damaging to the teeth.

Further, it should be noted that the effervescent combinations of acid and carbonate presented above leave a pleasant, clean feeling on the tooth surface and in the mouth.

In summary, it can be seen that a carefully balanced effervescent tablet is a novel base for delivering functional materials to the oral cavity. The inventive product avoids the use of abrasive and potentially gritty materials such as silica, silicon dioxide, aluminosilicate or calcium phosphate. Further, the consumer will enjoy using the product because the taste will be acceptable.

Although the composition is described as a tablet, the composition can be also be in particulate form (e.g., a powder).

Compositions as described herein are preferably substantially free of insoluble particulate material (e.g., abrasive agents including silica, silicate, aluminosilicate, or calcium phosphate). For example, compositions as described herein may contain less than 1 percent by weight of insoluble particulates. Alternatively, compositions as described herein can contain less than 0.5 or 0.1 percent by weight of insoluble particulates.

While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A composition comprising an effervescent combination of an acid and a carbonate salt dissolved in water, wherein the composition has an osmolality of 310 mOsm/kg of water or less.

2. The composition of claim 1, wherein the weight ratio of carbonate to acid is greater than 0.6.

3. The composition of claim 1, wherein the weight ratio of carbonate to acid is in the range of 1.25 to 4.5.

4. The composition of claim 1, wherein the effervescent acid is selected from the group consisting of: citric acid; fumaric acid; tartaric acid; malic acid; and adipic acid.

5. The composition of claim 1, wherein the effervescent acid is citric acid.

6. The composition of claim 1, wherein the carbonate salt is selected from the group consisting of: sodium, potassium, ammonium, magnesium, and calcium organic carbonate salts.

7. The composition of claim 1, wherein the carbonate salt is potassium carbonate, potassium bicarbonate, sodium carbonate or sodium bicarbonate.

8. The composition of claim 1, wherein the composition further comprises binders, fillers, and/or lubricants.

9. The composition of claim 1, wherein the effervescent acid is citric acid and wherein the carbonate salt is sodium bicarbonate.

10. The composition of claim 1, further comprising a flavoring agent.

11. The composition of claim 1, further comprising a binder.

12. The composition of claim 1, further comprising a functional additive selected from the group consisting of: anhydrous surfactants; anticaries ingredients;
bleaching agents; enzymes; desensitizing agents; antimicrobial agents; breath freshening ingredients; medicinal agents; dyes; and combinations thereof.

13. The composition of claim 1, wherein the effervescent acid and the carbonate salt make up at least 60 percent of the non-water weight of the composition.

14. The composition of claim 1, wherein the composition has a pH of less than 7.

15. The composition of claim 1, wherein the composition has a pH of from 4 to 7.

16. The composition of claim 1, wherein the composition has a pH of from 5 to 6, inclusive.

17. The composition of claim 1, wherein the weight ratio of carbonate to acid is in the range of 1.0 to 3.5.

18. The composition of claim 1, wherein the weight ratio of carbonate to acid is in the range of 1.25 to 2.5.

19. A method comprising:
dissolving a solid composition comprising an effervescent acid and a carbonate salt in water to form an effervescent solution; and
contacting the effervescent solution with oral tissue of a subject;
wherein the solution has an osmolality of 310 mOsm/kg or less.

20. The method of claim 19, wherein:
0.25 to 1.0 grams of the solid composition is dissolved in 15 to 25 ml of water.

21. The method of claim 19, wherein the solid composition is in tablet form.

22. The method of claim 19, wherein the solid composition has a moisture content of less than 1% by weight.

23. The composition of claim 19, wherein the solid composition has a moisture content of less than 0.2% by weight.

24. A method comprising:
placing a solid composition comprising an effervescent acid and a carbonate salt into an oral cavity of a subject; and
allowing the composition to dissolve and effervesce in the oral cavity;
wherein the weight ratio of carbonate to acid is in the range of 1.25 to 4.5; and
wherein the composition is substantially free of insoluble particulate material.

25. The method of claim 24, wherein between 0.25 grams and 1.0 gram, inclusive, of the composition are placed into the oral cavity.
